(19) United States
(12) Patent Application Publication
(10) Pub. No.: US 2015/0018792 A1
(43) Pub. Date: Jan. 15, 2015

(54) Wound Dressing Provided with a Detection System

(71) Applicant: COMMISSARIAT A L'ENERGIE ATOMIQUE ET AUX ENERGIES ALTERNATIVES, Paris (FR)

(72) Inventors: Cyril Marsiquet, Roumazieres Laubert (FR); Frederic Revol-Cavalier, Seyssins (FR)

(21) Appl. No.: 14/375,688

(22) PCT Filed: Jan. 29, 2013
(86) PCT No.: PCT/IB2013/050747

§ 371 Date: Jul. 30, 2014

(30) Foreign Application Priority Data
Jan. 30, 2012 (FR) 1250847

(51) Int. Cl. A61F 13/00

(52) U.S. Cl. CPC A61F 13/00055; A61F 13/00068; A61F 13/00042
USPC 604/361

(57) ABSTRACT

The present invention relates to a wound dressing comprising: —an application surface for application to a wound; —an absorbent structure for absorbing a liquid discharged from the wound; —an intermediate structure located between the application surface and the absorbent structure and arranged to promote the distribution of the liquid from the application surface to one or a plurality of inlet zones having a limited area in the absorbent structure; and —a detection system sensitive to the extent of the absorbent structure that is wetted by the liquid having penetrated same via the inlet zone or zones.

The present invention relates to dressings and systems used to improve the comfort of patients to whom these dressings are applied.

In Europe, the number of chronic wounds is constantly increasing and in most such cases hospitalization is required. The latter is accompanied by very high medical care and health costs.

At the present time, dressings are changed by medical personnel at regular intervals and independently of their level of saturation.

Thus, in certain cases, dressings are changed when they could have been left in place, thereby needlessly wasting human resources.

One approach allowing this problem to be solved consists in instrumenting the dressing in order to follow in real time not only the course of healing but also that of the dressing itself.

Instrumenting a dressing with a saturation sensor makes it possible to optimize when the dressing is changed and to help medical personnel organize their time.

The article David McColl, Brian Cartlidge, Patricia Connolly, Real-time monitoring of moisture levels in wound dressings in vitro: An experimental study. International Journal of Surgery (2007) 5, 316e322 reports a measurement method capable of following the course of the moisture level in a dressing in real time.

The principle used is the measurement of electrical impedance, allowing ionic liquids to be detected. The higher the volume of liquid in the dressing, the lower the impedance. A plurality of pairs of electrodes are distributed over the wound/dressing interface and thus allow a map of the moisture level in the dressing to be obtained.

However, this measurement has a drawback, namely that it is carried out uniquely at the wound/dressing interface. Thus, the moisture level measured may not correspond to that in the absorbent portion of the dressing.

The publication WO 99/17692 describes a saturation sensor with an indicator, this sensor being intended for use in absorbent tampons for feminine hygiene. This saturation sensor is formed by a plurality of wetness detectors distributed along the length of the tampon. Each detector is composed of two strips and of an absorbent substance placed between said two strips. The detector becomes conductive when the absorbent substance is wetted. Since the measurement of the wetness level is a point measurement, this detection system assumes that the migration of the body fluid occurs along the longitudinal axis of the tampon and uniformly.

The invention aims to further improve dressings by allowing their level of saturation to be determined.

Thus, the invention relates to a dressing comprising:
a face for application to a wound;
an absorbent structure for absorbing a liquid discharged from the wound;
an intermediate structure located between the application face and the absorbent structure and arranged to promote diffusion of the liquid from the application face toward one or more admission zones, of restricted extent, of the absorbent structure; and
a detection system sensitive to the extent of the absorbent structure wetted by the liquid having penetrated into said structure via the aforementioned admission zone(s).

The expression “of restricted extent” in the phrase “zone of restricted extent” is understood to mean that the extent of the zone is smaller than that of the absorbent structure.

In contrast to the measurement of the moisture level carried out by McColl where the electrodes did not form part of the absorbent structure and the publication WO 99/17692 which measured the wetness level only at precise locations in the tampon, the invention allows the saturation of the absorbent structure to be detected areally and the course of the wet area over time to be known. The area measured as wet corresponds to the actual area of the absorbent structure that is wet.

The intermediate structure preferably comprises a film forming a barrier to the liquid, which film is pierced with one or more openings in correspondence with said admission zone(s), especially between 1 and 10 openings. As a variant, or additionally, the intermediate structure promotes diffusion of the liquid toward one or more admission zones by virtue of an anisotropy in the one or more materials from which it is formed or of a treatment, for example impregnation, that blocks the pores on the face of the intermediate structure opposite the absorbent structure. The pores of the absorbent structure may also be obstructed on its face turned toward the wound, except in the admission zone(s).

The or all of said admission zones preferably correspond to less than 20% of the total area of the absorbent structure, in particular less than 5% of the total area of the absorbent structure.

The film forming a barrier, when present, is preferably hydrophobic. The film forming a barrier is preferably made of a semi-permeable material, being, for example, made of polyurethane. The term “semi-permeable” is understood to mean a material permeable to a gas, such as air, and impermeable to a liquid. Thus, the dressing preserves its breathability.

The detection system preferably comprises at least one network of electrodes, especially two networks of electrodes arranged in the form of a grid. The network(s) of electrodes are electrically insulated from one another, when the electrodes cross. When the detection system comprises two networks of electrodes arranged in the form of a grid, row and column matrices may be constructed with the results of measurements between consecutive pairs of electrodes of each network, and a matrix multiplication of the row and column matrices carried out to obtain a resultant matrix the coefficients of which are representative of the degree of wetness at various locations of the absorbent structure the coordinates of which are associated with the coefficients of the row and column matrices.

It is then possible to generate an item of information signaling not only the preference of wetness or not at such a location but also to quantify the degree of wetness at each location, which may be used as the basis for a cartographic representation in which the degrees of wetness are signaled by different colors.

The electrodes may be borne by a carrier that does not hinder the diffusion of the liquid and that is nonocclusive. This carrier may, for example, be a hydrophobic film, such as for example a hydrophobic nonwoven film. This makes it possible to prevent the liquid from spreading between the various electrodes.

The carrier of the electrodes may, if required, be the film forming a barrier or the absorbent structure itself, or an outer cover of the dressing.

The arrangement of the electrodes may be such that it is not necessary to place the electrodes on a carrier, which is for example the case when the electrodes are arranged in a grid.

The detection system preferably comprises a plurality of measuring locations distributed over the absorbent structure, so as preferably to obtain between 2 and 100 measurement locations, in particular from 10 to 50 measurement locations and more particularly from 30 to 50 measurement locations. A measurement location may be localized between two consecutive electrodes of the network of electrodes. The measurement may be carried out over substantially the entire length of these electrodes making contact with the absorbent structure.

The measurement locations may or may not be distributed uniformly over the absorbent structure. It may prove to be preferable to place measurement locations more closely together when far from the admission zone(s), in order to benefit from a better spatial resolution far from the admission zone(s) and thus to increase precision. For example, the gap between the electrodes of the network is smaller at distance from the admission zone(s) than nearby. The electrodes may or may not be rectilinear. For example, in the case of an arrangement in the form of a grid, at least certain electrodes may be curvilinear and bypass the admission zone(s).

The electrodes may be wires or produced by screen printing, etching or metallization. When the electrodes form a grid, they may be joined to one another at their intersections by an electrical insulator that fastens them together.

The detection system may be connected to a measurement system that for example determines the impedance between two consecutive electrodes, for at least a plurality of the consecutive pairs of electrodes that it is possible to generate. For example, the measurement system injects an alternating voltage between two consecutive electrodes and measures the amplitude of the current flowing in these electrodes, this current for example being determined by measuring the peak voltage across the terminals of a resistor passed through by this current. The detection system may be connected to the measuring system by one or more connections, in such a way that the measurement system is easily disconnectable from the detection system.

The alternating voltage is for example a low-frequency signal, of frequency lower than 25 kHz. The signal is for example a square waveform. The amplitude of the injected voltage is for example 5V or less.

The detection system preferably covers a detection area that corresponds to at least 50% of the total area of the absorbent structure.

The detection system may make contact with one face of the absorbent structure, which face is located opposite the intermediate structure. As a variant, the detection system makes contact with one face of the absorbent structure on the same side as the intermediate structure. These two variants may be combined, the detection system then being located on both sides of the absorbent structure.

The aforementioned admission zone(s) preferably occupy a central position relative to the absorbent structure. As a variant, the admission zone(s) occupy an off-center position relative to the absorbent structure. For example, the dressing comprises a single zone where liquid enters into the absorbent structure, which zone is located near its periphery, for example in a corner for a dressing of polygonal, especially square, outline, or as a variant as many admission zones as there are corners, each zone arranged in a corner.

The absorbent structure may be entirely or partially formed from a cellular material, especially based on an open cell polyurethane foam and/or from a fibrous material, for example based on cellulose fibers.

The absorbent structure may comprise one or more superabsorbent agents, for example polyacrylates.

The dressing according to the invention may comprise, between the face for application to the wound and the absorbent structure, a structure draining the liquid discharged from the wound.

The draining structure may comprise, even be formed from, two superposed layers, namely a proximal layer on the side of the wound, draining the liquid axially, and a distal layer which is superposed thereon, draining the liquid transversely.

The proximal layer draining the liquid axially may be entirely or partially formed from a cellular material, such as an open cell polyurethane foam for example.

The distal layer draining the liquid transversely may be entirely or partially formed from a nonwoven material or a knit, especially from viscose rayon.

The invention also relates to a method for estimating the degree of saturation of the absorbent structure of a dressing according to the invention, such as defined above, in which (i) by virtue of the detection system the extent of the absorbent structure wetted by the liquid discharged from the wound is detected; and (ii) depending on this extent the degree of saturation of the absorbent structure is determined.

The method may comprise displaying a quantity representative of the level of saturation and/or triggering an alarm when the level of saturation reaches a preset limit.

The level of saturation may correspond to the ratio of the extent detected as wet to the total extent of the absorbent structure; in this case, a location is considered to be wet if the measurement at this location gives a measurement that exceeds a preset threshold.

As a variant, the level of saturation is based on a quantitative measurement at each location of the degree of wetness, and the level of saturation is calculated from values measured at each location, for example peak voltages measured across the terminals of the aforementioned resistor.

The method may comprise the step consisting in measuring a quantity representative of the electrical impedance between two electrodes of the detection system, using a measurement device connected to the latter, especially to two consecutive electrodes.

Step (ii) may be repeated in order to measure said electrical impedance in succession between various successive pairs of electrodes of the or each network of electrodes.

The measurement system may be external to the dressing and the connections with the detection system may be established before a possible dressing change. As a variant, the measurement system is integrated into the dressing, by virtue for example of an electrical circuit to which the electrodes are connected, which performs the measurements at the terminals of the electrodes. The electronic circuit may transmit the results of the measurement to a display device external to the dressing, for example permanently present in the same room as the patient. As a variant, the electronic circuit comprises an RFID chip that is interrogated by the medical practitioner when the latter is present beside the patient. In this case, the power required by the measurement system to operate may be delivered via inductive coupling by the reader used by the medical practitioner.

In the case where the measurement system is an external system, the dressing may comprise an identifier that is detected by the measurement system. This allows the latter to select for example stored data tailored to the identified dressing, for example relating to the detection system present and the nature of the absorbent structure, in order to allow the measurement to take account thereof.

The measurement system may also be arranged to verify, before any measurements are carried out, that the absorbent structure is indeed dry and that the detection system has not been damaged. If required, measurements are carried out in order to serve as a reference later on.

By virtue of knowledge of the course of the degree of saturation of the absorbent structure, the amount of exudates received by the dressing may be evaluated.

The invention will be better understood on reading the following detailed description of nonlimiting example embodiments thereof, and on examining the appended drawings in which:

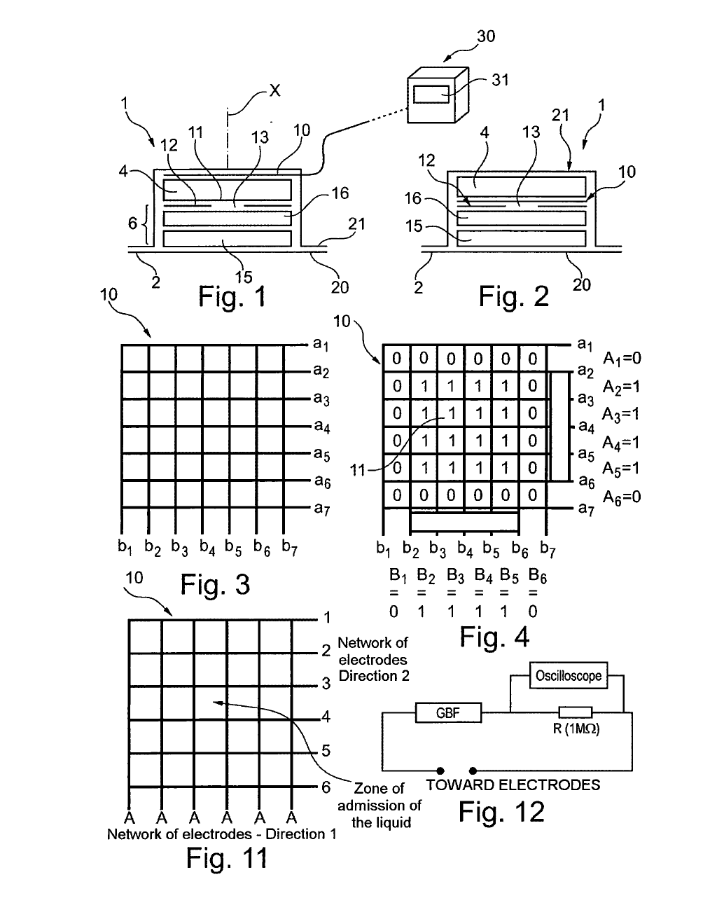

FIG. 1 is a schematic cross-sectional view of an example dressing according to the invention, connected to an external measurement system;

FIG. 2 is an analogous view to FIG. 1 of a variant embodiment of the dressing;

FIG. 3 shows an example arrangement of electrodes in a detection system;

FIG. 4 illustrates the use of the detection system in FIG. 3 to determine the extent of the wetted area;

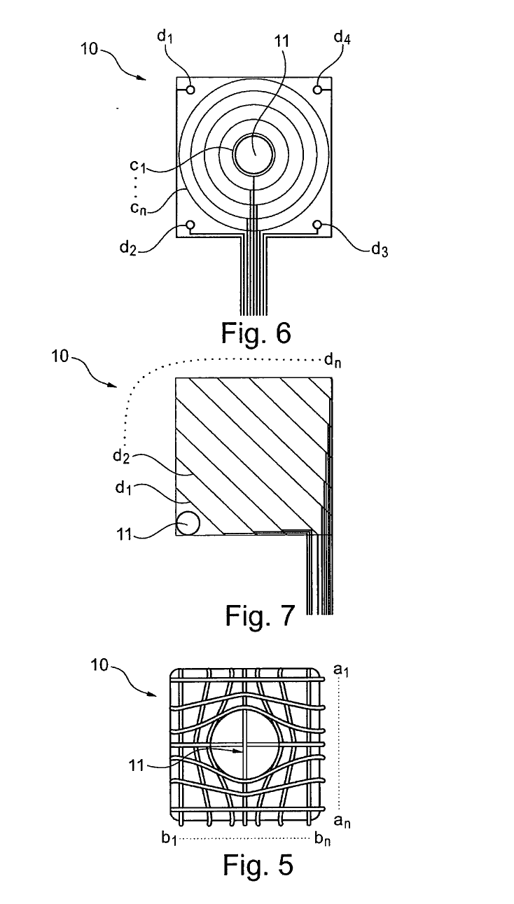

FIGS. 5 to 7 are analogous views to FIG. 3, of variant embodiments of the detection system;

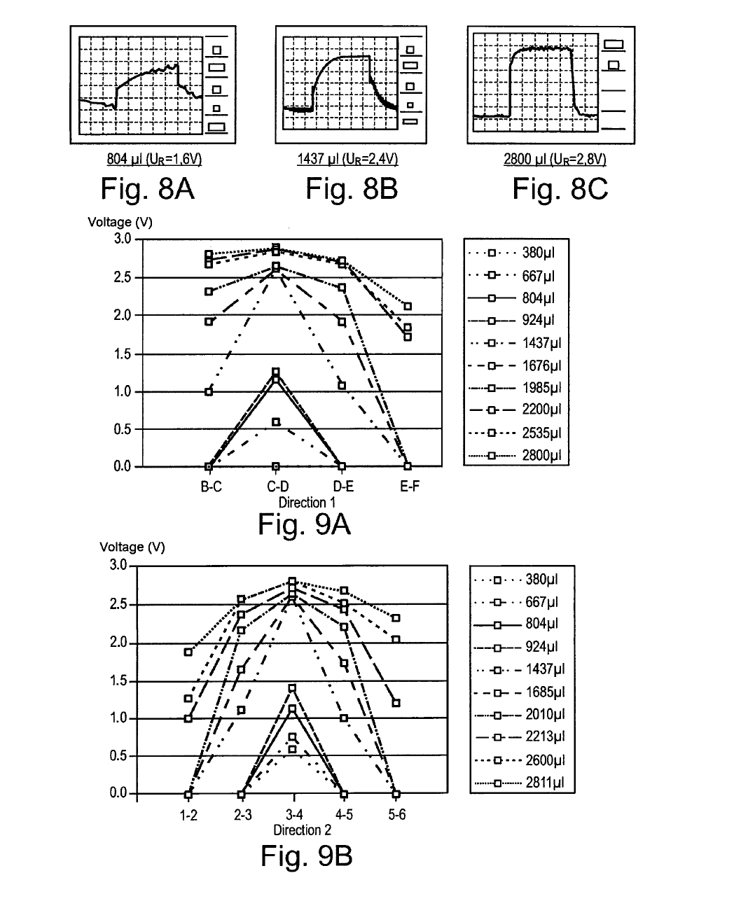

FIGS. 8A to 8C are oscillograms recorded during measurements during a trial;

FIGS. 9A and 9B show the variation in the voltage for various pairs of electrodes as a function of the degree of saturation of the absorbent structure, during the trial;

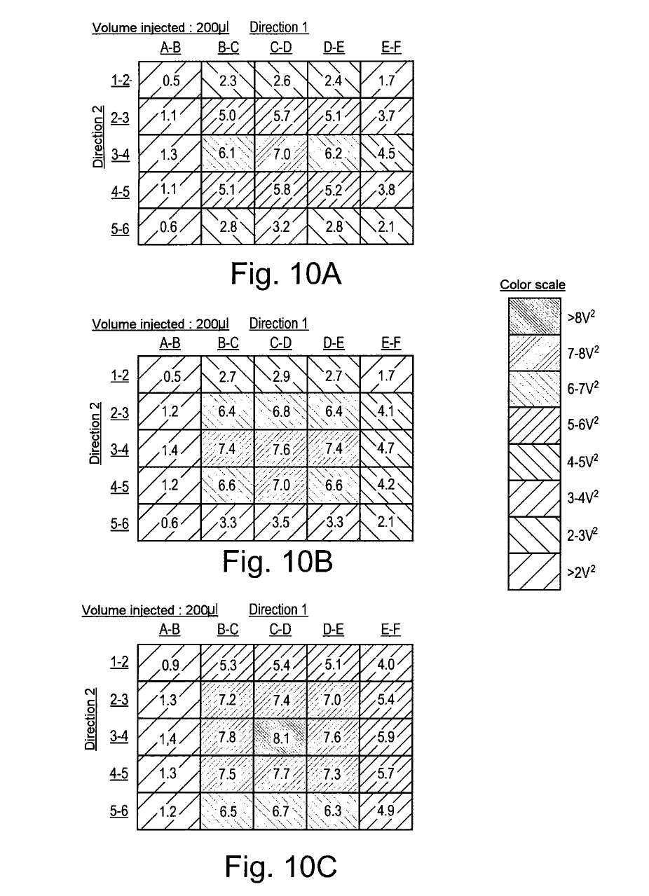

FIGS. 10A to 10C illustrate with colors the course of the saturation during the trial;

FIG. 11 shows an example detection system used for the trial; and

FIG. 12 is a schematic of a measurement system used for the trial of the detection system in FIG. 11.

In FIGS. 1 and 2, the actual proportions of the various constituent elements have not always been respected, for the sake of clarity of the drawing.

FIG. 1 shows an example dressing 1 according to the invention, connected to a measurement system 30 that for example comprises a screen 31 allowing information relating to the use of the dressing, especially its level of saturation, to be displayed.

The dressing 1 is contained in a sterile package before its first use.

In the example illustrated, the dressing 1 is connected by a wired connection to the measurement system 30, but as a variant information is transmitted by a wireless connection between the dressing 1 and the measurement system 30. In this case, the dressing is equipped with an electronic circuit that performs the measurements and transmits them to the measurement system, which may carry out some of the calculations or have only a display function. The measurement system may also optionally deliver, via inductive coupling, the power required by the electronic circuit to operate.

The dressing 1 has a face 2 for application to the wound and incorporates an absorbent structure 4 provided to absorb the liquid discharged by the wound.

The dressing 1 also comprises an intermediate structure 6 located between the application face 2 and the absorbent structure 4.

A detection system 10 is located making contact with the absorbent structure 4, for example above the latter, as illustrated in FIG. 1.

The detection system 10 is sensitive to the extent of the absorbent structure wetted by liquid having penetrated into said structure.

In order to promote diffusion of the liquid from the application face 2 toward an admission zone 11, of restricted extent, of the absorbent structure, the intermediate structure 6 comprises, in the example illustrated, a film 12 forming a barrier to the liquids, which film is arranged in contact with the lower face of the absorbent structure 4.

This film 12 forming a barrier is apertured opposite the admission zone 11 and thus contains at least one opening 13. The term “opening” is understood to mean a zone allowing the passage of a liquid from each side of the film 12 forming a barrier to the other. It may be a question of a hole or of a porous zone. It will be understood that in these alternatives, the liquid is able to pass from each side of the film 12 forming a barrier to the other.

The intermediate structure 6, between the film 12 forming a barrier and the application face 2, may comprise a draining structure composed of a proximal layer 15 draining the liquid axially, i.e. substantially along the X-axis perpendicular to the wound, vertical axis in FIG. 1, and a distal layer 16 draining the liquid transversely, i.e. substantially perpendicularly to the X-axis, i.e. horizontally in FIG. 1.

The proximal layer 15 is preferably, as illustrated, located adjacent the application face 2. The latter may be defined by a contact pad 20 of a material designed to make contact with the wound, especially a nonwoven material.

The dressing may comprise an outer cover 21 that is fixed to the pad 20 and that covers the layers 15 and 16, the film 12, the absorbent structure 4 and the detection system 10. The cover 21 may be made of a semi-permeable material, allowing air to pass.

The cover 21 may be fixed to the pad 20 on the periphery of the dressing 1, for example by welding or adhesive bonding.

The proximal draining layer 15 may be made of a cellular material, for example an open cell polyurethane foam.

The distal draining layer 16 is for example made of what is called a nonwoven material, especially of cellulose, and is for example made only of cellulose.

When the dressing 1 is in place on the wound, the liquid that rises into the intermediate structure 6 penetrates into the absorbent structure 4 locally through the aperture 13. Thus, admission of the liquid occurs via the zone(s) 11 opposite the aperture 13.

In the example in FIG. 1, the detection system 10 does not make contact with the film 12 since it is located between the absorbent structure 4 and the top of the cover 21. As a variant, the detection system 10 is located making contact with the film 12, between the latter and the absorbent structure 4, as illustrated in FIG. 2. In this case, the film 12 may serve as a carrier for the electrodes of the detection system, and for example receive conductive tracks deposited by printing or selective metallization. Especially in the case where the electrodes are printed and the latter cross, an insulator may be deposited locally at the intersections between the electrodes.

In the case where the detection system is coupled to a measurement system integrated into the dressing, an antenna may also be produced on the carrier of the electrodes. The detection system may be coupled to the measurement system by means of connectors arranged on the electrodes, the latter enabling reversible connection to the measurement system.

The position of the detection system may be chosen, if required, depending on the nature of the material forming the absorbent structure, so as to obtain the best results and/or the easiest manufacture. Thus, the two networks of electrodes may advantageously be located on either side of the absorbent structure, respectively; this embodiment allows the distribution of the liquid at various locations in the absorbent structure to be estimated.

The advantage of having a central aperture 13 consisting of one or more closely spaced holes is to obtain a concentric progression of the liquid within the absorbent structure 4 as it moistens, thereby decreasing the risk of generating erroneous information on the level of saturation of the absorbent structure.

The detection system 10 is produced so as to allow measurements to be taken at a plurality of locations, in order to determine the extent of the absorbent structure wetted by the liquid.

In order to allow measurements to be taken at a plurality of locations, the detection system may comprise at least one network of electrodes, each electrode occupying a known position relative to the admission zone(s) 11 via which the absorbent structure 4 fills with liquid on account of the presence of the aperture 13.

Thus, the distance from the electrodes of the network to each admission zone 11 varies uniformly or nonuniformly. The gap between electrodes within a network may vary and for example decrease with distance from an admission zone 11, in order to benefit from a better precision when the level of saturation of the absorbent structure is close to maximum.

Preferably, the detection system 10 comprises at least two networks of electrodes that are distributed along two different directions in the plane in which the absorbent structure 4 extends, for example two directions that are perpendicular to each other.

The presence of two networks of electrodes aᵢ and bⱼ arranged in a grid is particularly advantageous in that it allows a high number of measurement locations to be obtained, but the invention is not limited to any particular arrangement of the electrodes, provided that it is possible to obtain information on the extent of the absorbent structure 4 wetted by interrogating the detection system 10. The expression “measurement location” is understood to mean a zone of the absorbent structure 4 bounded by electrodes b_{b}. In the example considered, each measurement location corresponds to one cell of the grid.

FIG. 3 shows an example detection system 10 comprising networks of electrodes aᵢ and bᵢ arranged in a grid. In this figure, the detection system comprises a first network of electrodes a₁, . . . aᵣ, and a second network of electrodes b₁ . . . bₙ, where n is for example as illustrated equal to seven.

The electrodes are electrical conductors that cross without touching but that make electrical contact, at a plurality of points along their length, with the absorbent structure 4. When the absorbent structure 4 is permeated locally with liquid between two adjacent electrodes aⱼ, aⱼ₊₁, the impedance between these electrodes changes and this change may be detected by measuring an electrical quantity at the terminals of the electrodes. The same is true when two adjacent electrodes bⱼ, bⱼ₊₁ cover a zone locally permeated with liquid.

By way of example, in FIG. 4, the state of measurements carried out between various pairs of electrodes has been shown, the aperture 13 here consisting of four central holes defining as many admission zones 11.

The evaluation of the saturation of the absorbent structure 4 may consist, as in this example, in measuring the variation in the electrical conductivity between two successive electrodes aᵢ or bⱼ. In one example, when two successive electrodes make electrical contact because of the presence of liquid absorbed by the absorbent structure 4 between them, the parameters Aᵢ or Bⱼ are set to 1. The level of saturation of the absorbent structure may then be estimated by multiplying the column matrix A(A1, A2, A3, A4, A5, A6) by the row matrix B(B1, B2, B3, B4, B5, B6).

An example estimation of the level of saturation is shown in FIG. 4, where because of the presence of liquid between the electrodes a₂, a₃, a₄, a₅ and a₆ a column matrix A(A1, A2, A3, A4, A5, A6) equal to (0, 1, 1, 1, 1, 0) is obtained and because of the presence of liquid between the electrodes b₂, b₃, b₄, b₅ and b₆ a row matrix B(B1, B2, B3, B4, B5, B6) equal to (0, 1, 1, 1, 1, 0) is obtained.

The saturation of the absorbent structure is evaluated in this example on the basis of a wetness matrix C(i,j), determined by multiplying the column matrix A by the row matrix B. The coefficients of the matrix C(i,j) that are equal to 1 each represent a zone of the absorbent structure that is wet, and those that are equal to 0 a zone of the absorbent structure that is still dry.

On the basis of the zones measured as wet, the level of saturation may be visualized in a number of ways, using the measurement system 30 to which the detection system 10 is connected.

The result may be expressed in the form of a displayed value indicating the level of saturation of the absorbent structure 4, for example in percent, the value 100% corresponding to a dressing completely saturated with liquid. A map may also be displayed, showing, of all of the zones of the absorbent structure subjected to detection, those zones of the absorbent structure that are saturated.

The measurement system may be arranged, if required, to carry out supplementary measurements such as for example to indicate the time passed since the dressing was applied, the flow rate of liquid reaching the wound and the estimated time before the dressing needs to be changed.

Trial

The dressing is produced in the configuration in FIG. 1, by producing the interface pad 20 for contact with the wound from a viscose rayon/polyester nonwoven, the proximal draining structure 15 from a polyurethane foam and the distal draining structure 16 from a polyester/viscose rayon blend, in order to obtain a uniform horizontal distribution of exudates in this structure.

The film 12 forming a barrier is produced from a polyurethane film perforated at its center 4 with holes of 5 mm diameter. The film 12 is hydrophobic in order not to promote the spreading of body fluid.

The absorbent structure 4 is produced from polyurethane foam, or from cellulose fibers, and may be with or without superabsorbent such as polyacrylates.

The outer cover 21 is for example produced from a polyurethane film that is permeable to water vapor.

In the trial, the detection system 10 is formed from a superposition of two perpendicular networks of electrodes forming a grid, as illustrated in FIG. 11. These electrodes are all insulated from one another, when the dressing is dry. The electrodes may be insulated where they cross by an adhesive material such as Kapton™ tape, thereby allowing a self-supporting electrode assembly to be obtained, i.e. the electrodes of one network are kept in their relative positions by the electrodes of the other network, and vice versa. In addition, the detection system formed in this way is quite flexible, thereby allowing it to easily conform to the area to which it is applied.

The gap between the electrodes of each of the networks is for example 15 mm, the width of an electrode is for example 2 mm and the size of the grid is for example 70 mm by 70 mm.

The dressing is tested on a testbed simulating a wound.

The wound is simulated by a glass fit into which a model exudate solution is injected, for example an aqueous solution containing 0.9% by weight NaCl.

This solution is injected using a syringe driver at a rate of 6 μl/min, which corresponds to a flow rate value similar to that of a real wound.

During the trial, the electrodes of each network of the grid are alternately connected to the measurement system shown in FIG. 12, which is composed of a function generator (GBF), a measurement resistor R, for example a 1 Mohms resistor, and an oscilloscope. The electrodes A-B, B-C, C-D, D-E and E-F of the network associated with the direction 1 and the electrodes 1-2, 2-3, 3-4, 4-5, 5-6 of the network associated with the direction 2 are then connected in succession.

The signal output by the function generator is a square wave signal having a frequency of 10 kHz and a peak voltage of 3 V.

Each measurement consists in connecting the terminals of the measurement system to a pair of successive electrodes of the detection system and in measuring the maximum voltage across the terminals of the resistor R under these conditions. These successive connections may be carried out automatically using electronic switches.

FIGS. 8A to 8C show, for three different delivered volumes, the voltages measured across the terminals of the resistor R when the electrodes 3 and 4 of the detection system in FIG. 11 are connected. It will be observed that, when the volume of liquid located between the electrodes increases, the voltage across the terminals of the resistor R also gradually increases. Each pair of electrodes does not act as an on/off switch. It will also be noted that for volumes of 804 and 1437 μl, the signal across the terminals of the resistor R is not a square wave. Said signal approaches a square wave signal as the amount of liquid injected between the electrodes increases.

FIGS. 9A and 9B show the variation of the maximum voltage across the terminals of the resistor R for each pair of electrodes and for five volumes of liquid delivered to the dressing. For a given pair of electrodes, for example the pair C-D of the detection system in FIG. 11, the voltage across the terminals of the resistor R increases as the amount of liquid contained in the zone of the absorbent structure located between this electrode pair increases.

It may be seen that in the trial performed the absorbent structure moistened concentrically, starting at the center of the dressing then reaching the exterior thereof.

The level of saturation may be estimated in the example by multiplying the column matrix A(A1, A2, A3, A4, A5, A6) by the row matrix B(B1, B2, B3, B4, B5, B6). Each Aᵢ or Bⱼ corresponds to the value of the maximum voltage (peak voltage) measured by the oscilloscope for each pair of electrodes. The values shown in the colored squares in FIGS. 10A to 10C are the product of voltage Aᵢ multiplied by voltage Bⱼ. A color scale is for example used to make the saturation of the absorbent structure more visible (for example the color red=very wet, the color gray=a dry or almost dry zone)

This color map shows the concentric moistening of the absorbent. This confirms the vertical and central migration of exudates originating from the draining structure into the absorbent structure.

Furthermore, it may be seen that for a delivered volume of 2800 μl the dressing is not completely saturated because all the colored zones are not red.

The invention is not limited to a detection system 10 having a particular structure.

One variant of the detection system 10 is thus illustrated in FIG. 5. The objective of the geometry shown in FIG. 5 is to provide a better estimation of the level of saturation when the latter is close to its maximum value. The principle consists in moving apart the electrodes of the admission zone, at the center of the grid, as though the electrodes had to bypass a virtual cylinder placed in the center of the grid. Only the two central electrodes are still rectilinear, and the gap between two successive electrodes of a given network of electrodes at the point where they intersect with that electrode of the other network which is still rectilinear is smaller.

The variant illustrated in FIG. 6 comprises a network of concentric electrodes c₁, . . . , cₙ arranged around the central admission zone 11, and peripheral electrodes d₁, . . . , d₄ that terminate in the vicinity of each of the corners of the absorbent structure 4. The measurement may be carried out between each pair cⱼ, cⱼ₊₁ of consecutive electrodes, and between the radially outermost electrode cₙ and each of the corner electrodes d₁ to d₄. The supply conductors of the electrodes c₁ to cₙ and d₁ to d₄ may be electrically insulated from the absorbent structure in order for the measurement to indeed be taken with the electrodes.

The variant illustrated in FIG. 7 is characterized by the fact that saturation occurs from a corner, the admission zone 11 being off center.

The electrodes d₁, . . . , dₙ are for example arranged, as illustrated, from this corner in the direction of the opposite corner, for example parallel to each other and parallel to the diagonal connecting the two other corners. Thus, the electrodes are oriented substantially transversely to the direction in which the liquid propagates in the absorbent structure during the saturation of the latter. The measurement may be carried out between each pair dⱼ,dⱼ₊₁ of adjacent electrodes.

The invention is not limited to the illustrated examples. For example, yet other arrangements of electrodes may be used.

The expression “comprising a” or “comprising one” must be understood to be synonymous with “comprising at least one”.

1.-21. (canceled)

22. A dressing comprising:
a face for application to a wound;
an absorbent structure for absorbing a liquid discharged from the wound;
an intermediate structure located between the application face and the absorbent structure and arranged to promote diffusion of the liquid from the application face toward one or more admission zones, of restricted extent, of the absorbent structure; and
a detection system sensitive to the extent of the absorbent structure wetted by the liquid having penetrated into said structure via the admission zone(s).

23. The dressing as claimed in claim 22, the intermediate structure comprising a film forming a barrier to the liquid, which film is pierced with one or more openings in correspondence with the admission zone(s).

24. The dressing as claimed in claim 23, wherein said film is pierced with between 1 and 10 openings.

25. The dressing as claimed in claim 22, the admission zone(s) corresponding to less than 20% of the total area of the absorbent structure.

26. The dressing as claimed in claim 22, the admission zone(s) corresponding to less than 5% of the total area of the absorbent structure.

27. The dressing as claimed in claim 23, the film forming a barrier being hydrophobic.

28. The dressing as claimed in claim 23, the film forming a barrier being made of a semi-permeable material.

29. The dressing as claimed in claim 23, the film forming a barrier being made of polyurethane.

30. The dressing as claimed in claim 22, the detection system comprising one or more networks of electrodes.

31. The dressing as claimed in claim 22, comprising two networks of electrodes (a₁, . . . aₙ; b₁, . . . , bₙ) arranged in the form of a grid.

32. The dressing as claimed in claim 22, the detection system comprising a plurality of measuring locations distributed over the absorbent structure.

33. The dressing as claimed in claim 32, the detection system comprising between 2 and 100 measurement locations.

34. The dressing as claimed in claim 22, the detection system covering a detection area that corresponds to at least 50% of the total area of the absorbent structure.

35. The dressing as claimed in claim 22, the detection system making contact with one face of the absorbent structure, which face is located opposite the intermediate structure.

36. The dressing as claimed in claim 22, the detection system making contact with one face of the absorbent structure, which face is located on the same side as the intermediate structure.

37. The dressing as claimed in claim 22, the admission zone(s) occupying a central position relative to the absorbent structure.

38. The dressing as claimed in claim 22, the admission zone(s) occupying an off-center position relative to the absorbent structure.

39. The dressing as claimed in claim 22, the absorbent structure being entirely or partially formed from a cellular material, and/or from a fibrous material, and optionally comprising one or more superabsorbents.

40. The dressing as claimed in claim 39, said cellular material being based on an open cell polyurethane foam.

41. The dressing as claimed in claim 39, said fibrous material being based on cellulose fibers.

42. The dressing as claimed in claim 39, said superabsorbents being polyacrylates.

43. The dressing as claimed in claim 22, comprising, between the face for application to the wound and the absorbent structure, a structure draining the liquid discharged from the wound.

44. The dressing as claimed in claim 22, the draining structure comprising two superposed layers, the proximal layer on the side of the wound draining the liquid axially and the distal layer which is superposed thereon draining the liquid transversely.

45. The dressing as claimed claim 44, the proximal layer being entirely or partially formed from a cellular material and/or the distal draining layer being entirely or partially formed from a nonwoven material or a knit.

46. The dressing as claimed in claim 45, the proximal layer being entirely or partially formed from polyurethane foam.

47. The dressing as claimed in claim 45, the distal draining layer being entirely or partially formed from viscose rayon.

48. A method for estimating the degree of saturation of the absorbent structure of a dressing comprising:
a face for application to a wound;
an absorbent structure for absorbing a liquid discharged from the wound;
an intermediate structure located between the application face and the absorbent structure and arranged to promote diffusion of the liquid from the application face toward one or more admission zones, of restricted extent, of the absorbent structure; and
a detection system sensitive to the extent of the absorbent structure wetted by the liquid having penetrated into said structure via the admission zone(s), in which:
(i) by virtue of the detection system the extent of the absorbent structure wetted by the liquid discharged from the wound is detected; and
(ii) depending on this extent the degree of saturation of the absorbent structure is determined.

49. The method as claimed in claim 48, in which the detection system comprises one or more networks of electrodes, and step (i) comprises measuring an electrical quantity representative of the electrical impedance between two electrodes of the detection system using a measurement system.

50. The method as claimed in claim 49, step (i) being repeated in order to measure said electrical quantity between various successive pairs of electrodes of the or each network of electrodes.

51. The method as claimed in claim 48, in which, by virtue of knowledge of the course of the degree of saturation, the amount of exudates received by the dressing is evaluated.
